(19) United States
(12) Patent Application Publication
(10) Pub. No.: US 2012/0022032 A1
(43) Pub. Date: Jan. 26, 2012

(54) CORTICOSTEROID COMPOSITIONS AND METHODS OF TREATMENTS THEREOF

(75) Inventors: Jiansheng Wan, Warren, NJ (US); David Monteith, Pittstown, NJ (US); Pranav Gupta, Short Hills, NJ (US); Samir Anupam Shah, Springfield, NJ (US); Robert L. Berger, Bound Brook, NJ (US); Joel Sequeira, Venice, FL (US)

(73) Assignee: Schering Corporation, Kenilworth, NJ (US)

(21) Appl. No.: 13/123,670

(22) PCT Filed: Oct. 8, 2009
(86) PCT No.: PCT/US09/59970

§ 371 Date: Oct. 10, 2011

Related U.S. Application Data
(60) Provisional application No. 61/104,512, filed on Oct. 10, 2008.

(51) Int. Cl. A61K 31/58; A61P 11/06; A61P 11/00

(52) U.S. Cl. 514/172

(57) ABSTRACT

This invention relates to steroidal solutions for the preparation of medicaments and drug products useful for treating diseases of the upper and lower airway passages. Various embodiments of the present invention provide compositions, compositions and dosage forms with mometasone furoate in a dissolved state that are suitable for inhalation and can be used for the treatment of diseases of the upper and/or lower airway passages.

### FIELD OF THE INVENTION

This invention relates to steroidal compositions for the preparation of medicaments and drug products useful for treating diseases of the upper and lower airway passages.

### BACKGROUND OF THE INVENTION

Upper and lower airway conditions such as inflammatory conditions which include allergic rhinitis and asthma, affect a large amount of the population. Corticosteroids have been approved to reduce inflammation of the upper and lower airways. For instance, intranasal corticosteroids exert a range of effects that inhibit mucosal inflammation, including (1) reducing inflammatory cell infiltration, (2) decreasing the number of basophils, eosinophils, neutrophils and mast cells in the nasal passages and their secretions, (3) reducing release of inflammatory signals from cells, (4) decreasing mucus production, (5) vasoconstriction and (6) reducing edema.

Although corticosteroids have been effective in treating airway passage diseases, such treating with corticosteroids may often cause systemic side-effects such as suppression of hypothalamic-pituitary-adrenocortical (“BPA”) axis function by reducing corticotrophin (ACTH) production, which in turn leads to a reduced cortisol secretion by the adrenal gland.

Many efforts have been put forth in designing a safe and efficacious steroidal composition. Several corticosteroids have been successfully formulated as aqueous suspensions. However, suspension compositions may not be completely desirable in some circumstances. Solution compositions may offer certain advantages under particular conditions. It has been thought that a steroidal solution composition would have an unacceptable safety profile because of increased systemic absorption which could suppress HPA axis function of patients. Thus, it would be desirable to provide a solution composition suitable for inhalation that includes a steroidal active pharmaceutical agent and has an acceptable safety and efficacy profile.

### SUMMARY OF THE INVENTION

Multiple embodiments of the present invention provide pharmaceutical compositions comprising a steroid solution suitable for inhalation, wherein the concentration of the steroid is from about 0.1 micrograms (mcg)/ml to about 500 mcg/ml. The solvent used for the solution may be aqueous or non-aqueous based. Suitable non-aqueous solvents include propellants such as CFC's or non-CFC's, such as 1,1,1,2 tetrafluoroethance (HFA 134) and 1,1,1,2,3,3,3 heptafluoroethane (HFA 227). Suitable steroids include but are not limited to mometasone furoate (MF), fluticasone propionate, fluticasone furoate, budesonide, triamcinolone acetonide, prednisolone, beclomethasone dipropionate, ciclesonide and flunisolide.

Several embodiments of the present invention provide solution compositions that may include a co-solvent system, a complexation system, a cyclodextrin system, or a lipid based system which may include an emulsion, microemulsion or micellar composition, that includes a steroid, such as MF, in dissolved form, in a therapeutically effective amount.

Various embodiments of the present invention provide for pharmaceutical compositions comprising a mometasone furoate aqueous solution suitable for inhalation, wherein the concentration of mometasone furoate is from about 0.1 mcg/ml to about 500 mcg/ml. Alternatively, the concentration of the mometasone furoate may be from about 5 mcg/ml to about 100 mcg/ml, from about 25 mcg/ml to about 75 mcg/ml, from about 50 mcg/ml to about 75 mcg/ml, from about 25 mcg/ml to about 50 mcg/ml, from about 60 mcg/ml to about 65 mcg/ml, or about 62.5 mcg/ml.

Various compositions of the present invention may include at least one co-solvent. The at least one co-solvent may be propylene glycol, polyethylene glycol 300, polyethylene glycol 400, ethanol and glycerol or a combination of two or more thereof. A particularly useful co-solvent is polyethylene glycol. The at least one co-solvent may be present in an amount from about 0.01 to about 60% by weight or from about 5 to about 15% by weight.

Various compositions of the present invention may comprise at least one surfactant or at least one surfactant and at least one oil. The surfactant may be present in an amount from about 0.01 to about 40% by weight or from about 1 to about 20%. The oil may be present in an amount from about 0.01 to about 40% by weight or from about 1 to about 20%.

Suitable oils include, but are not limited to, short, medium and long chain mono glycerides, diglycerides and triglycerides. Suitable oils include, among others, caprylic and capric acid triglyceride and useful surfactants include polyethylene glycol 660-12 hydroxystearate also known as macrogol-15-hydroxystearate and marketed with the trade name Solutol HS 15, and others etc.

Various compositions of the present invention may include at least one rheology-modifying agent. Suitable rheology modifying agents include, but are not limited to, sodium carboxymethyl cellulose, etc.

Various compositions of the present invention may include at least one additional active pharmaceutical agent (APA). Suitable additional APAs include decongestants, antihistamines, beta agonists and anticholinergics and combinations thereof. A particularly useful additional APA is a decongestant, such as oxymetazoline.

Other embodiments of the present invention provide pharmaceutical drug products that comprise an inhalation device and an aqueous solution suitable for inhalation comprising mometasone furoate in solution in a concentration from about 0.1 mcg/ml to about 500 mcg/ml. Other useful concentrations of MF may be from about 5 mcg/ml to about 100 mcg/ml or from about 25 mcg/ml to about 75 mcg/ml. Useful inhalation devices include a nasal spray, soft mist inhaler, pressurized metered dose inhaler; a nebulizer and the like. Another embodiment of the present invention provides a method of administering the drug product by applying the inhalation device to each nostril of the nose and actuating the inhalation device at least once to each nostril to deliver the solution to the nasal cavity.

Still other embodiments of the present invention provide methods of treating allergic rhinitis which comprise administering a mometasone furoate solution suitable for inhalation once daily to the upper airway passages; wherein the total daily dose of mometasone furoate is from about 0.04 to about 200 micrograms. Other suitable daily dose amounts of mometasone furoate include from about 5 to about 100 micrograms; from about 5 to about 50 micrograms; from about 10 to about 45 micrograms or from about 20 to about 25 micrograms. Such amounts are useful to treat seasonal or perennial allergic rhinitis.

Other embodiments of the present invention provide methods of treating nasal polyposis including administering a mometasone furoate solution suitable for inhalation once or twice daily to the upper airway passages; wherein the total daily dose of mometasone furoate is from about 5 to about 200 micrograms of mometasone furoate. Alternative useful total daily doses include from about 0.04 to about 100 micrograms of mometasone furoate, from about 40 to about 50 micrograms of mometasone furoate.

Additional embodiments of the present invention provide methods of treating an airway disease which comprises administering a mometasone furoate solution suitable for inhalation once daily to the upper or lower airway passages; wherein the total daily dose of mometasone furoate is from about 0.04 to about 200 micrograms. The total daily dose of mometasone furoate may be from about 5 to about 100 micrograms of mometasone furoate or from about 10 to about 50. Airway diseases that may be treated with this method include asthma, chronic obstructive pulmonary disease, sinusitis, allergic rhinitis and/or nasal polyposis and combinations thereof.

Still other embodiments provide methods of treating a corticosteroid-responsive disease of the upper or lower airway passages in patients afflicted with said disease, which comprises administering to the surfaces of the passages of the patients a therapeutically effective amount of a solution of mometasone furoate effective for treating the disease. The solution may be administered once a day and may contain from about 0.04 to about 200 micrograms of mometasone furoate, from about 0.04 to about 100 micrograms of mometasone furoate or from about 5 to about 50 micrograms of mometasone furoate.

Various other embodiments provide methods of administering a pharmaceutical composition that targets a mometasone furoate solution to provide a time to maximum plasma concentration (Tₘₐₓ) of mometasone furoate of less than one hour post dose. Suitable mometasone furoate concentration amounts include from about 0.1 mcg/ml to about 500 mcg/ml from about 5 mcg/ml to about 100 mcg/ml. The total daily dose of mometasone furoate administered may be from about 5 to about 50 micrograms and the composition may be administered once a day.

Various other embodiments provide pharmaceutical compositions that comprise an aqueous solution suitable for inhalation which comprises mometasone furoate; a surfactant; optionally an oil; and water. The solution may be a microemulsion or a micellar composition.

Multiple embodiments provide pharmaceutical compositions which comprise an aqueous solution suitable for inhalation that comprises mometasone furoate in a concentration from about 5 mcg/ml to about 100 mcg/ml; at least one surfactant in a concentration from about 0.01 to about 20%; optionally at least one oil in a concentration from about 0.01 to about 20%; and water. At least one rheology-modifying agent may also be included.

Other embodiments provide a pharmaceutical composition comprising an aqueous solution suitable for inhalation comprising mometasone furoate; at least one surfactant with a hydrophilic-lipophilic balance (HLB) value from about 3 to about 18, optionally at least one oil comprising a fatty acid carbon chain length of C₆-C₂₂ fatty acid; and water.

Other embodiments provide pharmaceutical compositions having an aqueous solution suitable for inhalation having mometasone furoate; at least one co-solvent; and water. Suitable co-solvents include propylene glycol, polyethylene glycol 300, polyethylene glycol 400, ethanol and glycerol and combinations thereof.

Still further embodiments provide pharmaceutical compositions having an aqueous solution suitable for inhalation, which comprises mometasone furoate; at least one excipient having a hydrophilic moiety and a hydrophobic moiety; and water. The excipient may be a cyclodextrin.

Additional embodiments provide a pharmaceutical composition comprising a solution suitable for inhalation which includes mometasone furoate; at least one propellant; at least one co-solvent; optionally at least one surfactant; wherein the concentration of mometasone furoate is from about 0.1 mcg/ml to about 500 mcg/ml or alternatively from about 5 mcg/ml to about 100 mcg/ml; from about 25 mcg/ml to about 75 mcg/ml; from about 60 mcg/ml to about 65 mcg/ml; from about 25 mcg/ml to about 50 mcg/ml; or about 62.5 mcg/ml. Suitable at least one co-solvent include but are not limited to propylene glycol, polyethylene glycol 300, polyethylene glycol 400, ethanol, N—N dimethylacetamide, N-methyl-2-pyrolidone and glycerol and combinations thereof. One particularly suitable co-solvent, among others, is ethanol, etc. The at least one co-solvent may be present in an amount from about 0.01 to about 60% by weight or from about 5 to about 15% by weight. The at least one surfactant may be present in an amount from about 0.01 to about 40% by weight or from about 1 to about 10% by weight. Another embodiment provides for a pharmaceutical drug product comprising a metered dose inhaler canister, valve and this composition. The at least one propellant may be 1,1,1,2 tetrafluoroethance (1-WA 134) and 1,1,1,2,3,3,3 heptafluoroethane (HFA 227) and combinations thereof.

### BRIEF DESCRIPTION OF DRAWINGS

FIG. 1 Comparison of average human plasma levels over time of the NASONEX® suspension nasal spray versus the MF microemulsion solution nasal spray when delivered from the same device at a 200 mcg dose

FIG. 2 MF of solubility of anhydrous and monohydrate forms of MF in microemulsions composed of 20% w/w Solutol HS 15 and various oils at different amounts.

FIG. 3 Solubility of MF with various cyclodextrins of varying conformation and substitution, versus concentration in 3 mM citrate Buffer at pH 4.5.

FIG. 4 The effect of additives on the solubility (mcg/ml) of MF in various 0.2M sulfobutyl ether beta cyclodextrin (CAPTISOL®) buffered solutions.

FIG. 5 Solubility of anhydrous MF (mcg/ml) in various excipients at 10% w/w and 90% water w/w

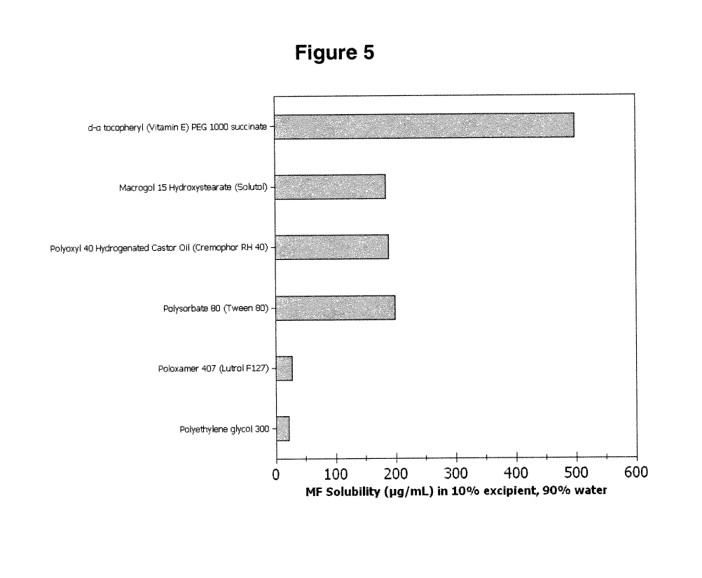

### DETAILED DESCRIPTION

Several embodiments of the present invention provide compositions that are steroidal solutions suitable for inhalation. Surprisingly, the compositions are able to provide aqueous or non-aqueous based solutions that include a poorly water soluble steroids in a therapeutically effective amount. The solvent used for the solution may be aqueous or non-aqueous based. Suitable non-aqueous solvents include propellants such as CFC's or non-CFC's, such as 1,1,1,2 tetrafluoroethance (HFA 134) and 1,1,1,2,3,3,3 heptafluoroethane (HFA 227). Suitable steroids include but are not limited to mometasone furoate (MF), fluticasone propionate, fluticasone furoate, budesonide, triamcinolone acetonide, prednisolone, beclomethasone dipropionate, ciclesonide and flunisolide. A particularly useful steroid is mometasone furoate.

Several embodiments of the present invention provide compositions including at least one APA, such as a corticosteroid, such as MF, in a dissolved state. Such compositions with at least one APA in a dissolved state are referred to as solutions. Compositions including at least one APA in a dissolved state may be prepared as a solution by any suitable method including, but not limited to, with the use co-solvents, complexation agents, cyclodextrins, or a solution may be a lipid based composition including, but not limited to an emulsion, microemulsion, or micellar solution.

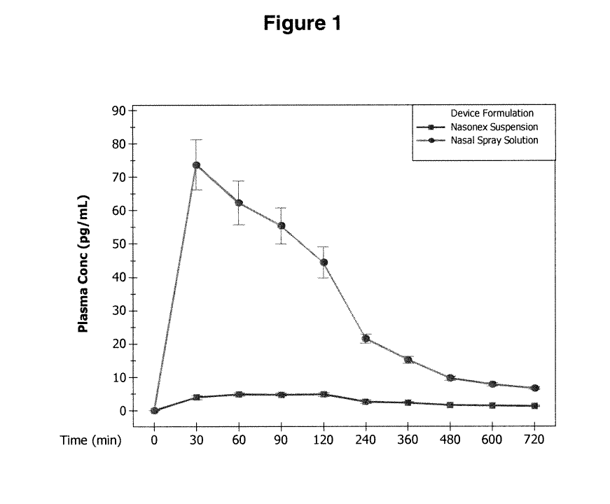

Presenting an APA, such as MF, in solution is provided by various embodiments of the present invention in which the drug is molecularly dispersed resulted in surprisingly highly absorbed drug products. Solutions containing MF show a surprisingly rapid increase in blood levels, see FIG. 1. Rapid absorption at the sites of inflammation may yield a good therapeutic effect to treat many upper and lower airway diseases such as nasal symptoms and non-nasal symptoms (including ocular redness, itching, and tearing, secretions in the throat, irritation of the throat, coughing, decreased hearing, popping, itching of the ears, headaches, and/or facial pressure). Increased absorption at the sites of inflammation may also reduce the onset of action time. The time to maximum benefit to the patient could also be reduced.

A lower spray amount may be used, which could reduce spray volume and lower the daily exposure to preservatives and other inactive ingredients in the composition. Additionally, a lower spray volume may reduce the sensation of “dripping”; reducing the patient's tendency to sniff, which reduces the retention time on the tissue making the drug appear to be less effective. Less dripping would also increase patient compliance among those who find this attribute undesirable.

Suitable solutions include co-solvent compositions where a co-solvent reduces the dielectric constant of water and facilitates hydrophobic interactions of drug molecules with the solvent system. Suitable co-solvents include, but are not limited to, organic solvents such as ethanol, propylene glycol and polyethylene glycol.

Suitable solutions may include a cyclodextrin, including but not limited to, hydroxypropyl-β-cyclodextrin (HPBCD), such as CAPTISOL®, sulfobutyl ether β-cyclodextrin.

Suitable solutions include emulsions and microemulsions, which are systems of water, at least one surfactant and at least one oil. Desirably they optically appear as a single phase.

Suitable solutions include micellar solutions, which are typically optically clear and thermodynamically stable and have enough surfactant to form aggregates with a lipophilic core and hydrophilic surface.

Compositions of multiple embodiments of the present invention may be prepared by using solutions that include polyethylene glycol, polyethylene glycol 12 oxy-stearate, d-alpha tocopheryl polyethylene glycol 1000 succinate, polyoxyethylene, sorbitan monooleate, macrogol hydroxystearate, poloxamers, ethyl laurate, and oils such as short, medium, and long chain monoglycerides, diglycerides and triglycerides and combinations thereof.

Suitable surfactants and/or solubilizers include, but are not limited to, medium chain mono- and diglyceride sold as IMWITOR(S)® by Sasol; distilled acetylated monoglyceride sold as MYVACET 9-45® by Eastman Chemical Company; long chain monoglyceride sold as PECEOL® (GLYCERYL MONOOLEATE), MAISINE® (GLYCERYL MONOLINOLEATE) (S) by Gattefosse; propylene glycol monocaprylate sold as CAPRYOL 90 (S)® by Gattefosse; propylene glycol caprylate sold as CAPYROL PGMC (S)® by Gattefosse; diethylene glycol monoethyl ether sold as TRANSCUTOL (S)® by Gattefosse; polyethylene glycol 660-12 hydroxystearate sold as SOLUTOL HS-15® by BASF; polyoxylglycerides sold as GELUCIRE® 33/01, 39/01, 43/01, 44/14, 50/13 by Gattefosse; polyoxyl 40 hydrogenated castor oil sold as CREMOPHOR RH40® by BASF; polyoxyl 35 castor oil sold as CREMOPHOR EL® by BASF; d-α-tocopheryl polyethylene glycol 1000 succinate sold as VITAMIN E TPGS® by Eastman Kodak; PEG 300 linoleic glyceride sold as LABRAFIL M-2125CS® by Gattefosse; PEG 400 caprylic/capric glyceride sold as LABRASOL® by Gattefosse; PEG 300 oleic glyceride sold as LABRAFIL M-1944CS® by Gattefosse; PEG 300 caprylic/capric glyceride sold as SOFTIGEN 767® by Gattefosse; polyethylene oxide/poly-(propyleneoxide)/poly (ethyleneoxide) triblock copolymers sold as Poloxamers/Pluronics by BASF; polyoxyethylene 20 sorbitan monooleate sold as TWEEN 20/TWEEN 80® (Polysorbate 20, Polysorbate 80) by Sigma; Sorbiton monooleate sold as SPAN 20/SPAN 80® by ICI Americas, Inc; macrogol-glycerolhydroxystearat (DAB) or polyoxyethylenglyceroltrihydroxystearat (DAC) and combinations thereof. Polyethylene glycol 660-12 hydroxystearate is a particularly useful surfactant.

Suitable concentration for surfactants will vary depending on the amount of other excipients and steroid used. Suitable surfactant amounts include from about 0.001% to about 80% by weight; from 0.01 to about 80% by weight; from about 0.01 to about 60% by weight; from about 0.01 to about 40% by weight; from about 0.01 to about 20% by weight; from about 1% to about 15% by weight; from about 1% to about 10% by weight; from about 1 to about 5% by weight or from about 0.01 to about 5% by weight.

MF is highly lipophilic. Excipients used to solubilize MF desirably have a lipophilic component to help it associate with MF. Surfactants are amphiphilic molecules having both lipophilic and hydrophilic moieties to form a hydrophobic core containing the oil and hydrophobic drug, when dispersed in water. If the surfactant concentration is greater than the critical micelle concentration, the lipophilic cores of the surfactant aggregate, forming a micelle, which encapsulates the lipophilic molecule. The hydrophilic components of the surfactant associate with the water.

In choosing a good surfactant, the percentage of hydrophilic versus lipophilic moieties on the molecules are desirably balanced. When evaluating 10% excipients in water, vitamin E TPGS solution was found to solubilize a high amount of MF. The lipophilic parts of the molecule included 12 carbon saturated alkyl chains and a benzene ring that could help solubilize the MF.

The hydrophilic-lipophilic balance (HLB) of a surfactant is a measure used to determine the degree to which the surfactant is hydrophilic or lipophilic. HLB values are determined by calculating values for the different regions of the molecule, as described by Griffin and the Davis method. Suitable surfactants typically will have HLB values from about 3 and above; from about 3 to about 18 or from about 8 to about 18. Oil/water emulsions can be formulated with surfactant/surfactant blend with HLB values from about 8 to about 18 and water/oil emulsions can be formulated with HLB values from about 3 to about 6.

Suitable medium chain triglycerides include, but are not limited to, caprylic and capric acid triglycerides sold as propylene glycol dicaprylate/dicaprate sold as MIGLYOL 840 by Sasol and CAPMUL 200 (S)® by Abitech; MIGLYOL 812/MIGLYOL 810@ by Sasol North America; of caprylic and capric acid linoleic acid triglycerides sold as MYGLYOL 818® by Sasol North America; trigylceride from coconut oil sold as CAPTEX 300/CAPTEX 850® by Abitech Corp; caprylic/caprylic triglyceride sold as CAPTEX 355® by Abitech Corp; caprylic/caprylic/lauric triglyceride sold as CAPTEX 350® by Abitech Corp; caprylic/caprylic/linoleic triglyceride sold as CAPTEX 810® by Abitech Corp; caprylic/caprylic/stearic triglyceride sold as CAPTEX SBE® by Abitech Corp; tricaprylic/Caprylic triglyceride ester sold as NEOBEE M-5® by Stephan and combinations thereof.

Suitable long chain triglyercerides include, but are not limited to, soybean oil sold as SUPER-REFINED SOYBEAN OIL USP® by Croda; corn oil sold as SUPER-REFINED CORN OIL NF® by Croda; cottonseed oil sold as SUPER-REFINED COTTONSEED OIL NF® by Croda; olive oil sold as SUPER-REFINED OLIVE OIL NF® by Croda; peanut oil sold as SUPER-REFINED PEANUT OIL BF® by Croda; safflower oil sold as SUPER-REFINED SAFFLOWER USP® by Croda; sesame oil sold as SUPER-REFINED SESAME NF® by Croda; shark liver oil sold as; SUPER-REFINED SHARK LIVER® by Croda; castor oil; monounsaturated omega-9 fatty acid sold as Oleic acid by Croda; peppermint oil; hydrogenated palm oil sold as SOFTISAN 154® by Sasol and combinations thereof.

Suitable concentrations for oils will vary depending on the amount of other excipients and steroid used. Suitable amounts include from about 0.001% to about 80% by weight; from 0.01 to about 80% by weight; from about 0.01 to about 60% by weight; from about 0.01 to about 40% by weight; from about 0.01 to about 20% by weight; from about 1% to about 15% by weight; from about 1% to about 10% by weight; from about 5 to about 10% by weight or from about 0.01 to about 0.25% by weight.

Suitable co-solvents include, but are not limited to, propylene glycol, PEG 300, PEG 400, ethanol, N—N dimethylacetamide (DMA), N-methyl-2-pyrolidone (NMP), glycerol and combinations thereof. Up to 55% of the solution may be the co-solvent. Preferably, the co-solvent is in the range from about 0.01 to about 60% by weight, from about 1 to about 20% by weight, from about 5 to about 20% by weight, from about 1 to about 10% by weight, from about 5 to about 15% by weight or from about 5 to about 10% by weight. Polyethylene glycol is a particularly useful solvent.

Cyclodextrins are cyclic carbohydrates derived from starch. The unmodified cyclodextrins differ by the number of glucopyranose units joined together in the cylindrical structure. The parent cyclodextrins contain 6, 7, or 8 glucopyranose units and are referred to as α-, β-, and γ-cyclodextrin respectively. Each cyclodextrin subunit has secondary hydroxyl groups at the 2 and 3 positions and a primary hydroxyl group at the 6 position. The cyclodextrins may be pictured as hollow truncated cones with hydrophilic exterior surfaces and hydrophobic interior cavities. In aqueous solutions, these hydrophobic cavities provide a haven for hydrophobic organic compounds that can fit all or part of their structure into these cavities. This process, known as inclusion complexation, may result in increased apparent aqueous solubility and stability for the complexed drug. The complex is stabilized by hydrophobic interactions and does not involve the formation of any covalent bonds.

Suitable cyclodextrins include those described in U.S. Pat. Nos. 5,376,645 and 5,134,127 to Stella et al, the entire disclosures of which are hereby incorporated by reference. The preparation process may comprise dissolving the cyclodextrin in aqueous base at an appropriate temperature, e.g., 70° to 80° C., at the highest concentration possible. For example, to prepare the cyclodextrin derivatives herein, an amount of an appropriate alkyl sultone, corresponding to the number of moles of primary CD hydroxyl group present, is added with vigorous stirring to ensure maximal contact of the heterogeneous phase. Suitable cyclodextrins include but are not limited to SBE-7-β-CD (CAPTISOL®), or SBE-4-β-CD available from Cyclex, Inc.

Mometasone solution compositions may be prepared by admixing mometasone furoate with water and other pharmaceutically acceptable excipients, see Example 1. Solution compositions may contain, inter alia, water, and/or one or more of the excipients, such as: suspending agents, e.g., microcrystalline cellulose, sodium carboxymethylcellulose, hydroxpropyl-methyl cellulose; humectants, e.g. glycerin and propylene glycol; acids, bases or buffer substances for adjusting the pH, e.g., citric acid, sodium citrate, phosphoric acid, sodium phosphate as well as mixtures of citrate and phosphate buffers; surfactants, e.g. polysorbate 80; and antimicrobial preservatives, e.g., benzalkonium chloride, phenylethyl alcohol and potassium sorbate. Combinations of any of these pharmaceutically acceptable excipients may be used also.

Additional embodiments of the present invention provide for methods of treating upper and lower respiratory diseases, such as seasonal and/or perennial allergic rhinitis, asthma, COPD, rhinosinusitis or nasal polyps, with a steroidal solution at surprisingly low dose amounts.

It was discovered that the systemic exposure of mometasone furoate from a mometasone furoate solution composition nasal spray in humans is about 9 to about 10 times greater than the systemic exposure from the NASONEX® mometasone furoate monohydrate suspension composition nasal spray when delivered at 200 mcg dose from the same type of device. The in vivo systemic exposure was determined by taking the geometric mean of the area under the curve (AUC) of the plasma levels at the time points measured for each subject. The magnitude of this difference in systemic exposure between an MF solution and an MF suspension is surprising when considered in view of previous studies conducted on other intranasal corticosteroids: (a) only a 1-2 times difference in relative bioavailability was seen between a suspension and solution of triamcinolone acetonide nasal spray (Hochhaus 2002, J Clin Pharmacol), and (b) only a 2.5 times difference in relative bioavailability was seen between a suspension and solution of beclomethasone dipropionate on oral inhalation (Vanden Burgt 2000 J Allergy Clin Immunol). Thus, the compositions of the present invention are surprising in view of previous corticosteroid solutions, which have been prepared but at higher total daily doses. Due to the dramatic increase in bioavailability of the solutions of various embodiments of the present invention, the total daily dose is surprisingly small.

FIG. 1 compares systemic exposure of MF following administration of the two compositions to healthy humans described in relation to time. The in vivo systemic exposure was determined by taking the geometric mean of the area under the curve (AUC) of the plasma levels at the time points measured for each subject. When comparing a NASONEX® suspension (upper line-circles) to a mometasone furoate solution, both at 200 mcg, (lower line-squares) the MF exposure from a mometasone furoate solution were significantly greater (about 9 to about 10 times) than from a NASONEX® suspension even though both compositions were delivered at the same dose and with the same type of device. At 12 hours after dosing, concentrations of MF after administration as a solution were still approximately 6 times higher than the MF concentrations observed after administration of MF in the form of a suspension. The time to maximum concentration (Tₘₐₓ) is less than one hour, which is surprisingly quick. The maximum concentration (Cₘₐₓ) is desirably from about 1 picograms(pg)/ml to about 75 pg/ml, from about 5 pg/ml to about 20 pg/ml; or about 5 pg/ml to about 10 pg/ml.

Suitable concentrations of mometasone furoate include from about 0.1 micrograms (mcg)/ml to about 500 mcg/ml; 1 mcg/ml to about 500 mcg/ml from about 5 mcg/ml to about 500 mcg/ml; 5 mcg/ml to about 250 mcg/ml; from about 5 mcg/ml to about 100 mcg/ml; from about 10 mcg/ml to about 100 mcg/ml; from about 50 mcg/ml to about 100 mcg/ml; from about 25 mcg/ml to about 75 mcg/ml; from about 50 mcg/ml to about 75 mcg/ml; from about 5 mcg/ml to about 50 mcg/ml; from about 60 mcg/ml to about 65 mcg/ml; about 5 mcg/ml; about 10 mcg/ml; about 15 mcg/ml; about 20 mcg/ml; about 25 mcg/ml; about 30 mcg/ml; about 35 mcg/ml; about 40 mcg/ml; about 45 mcg/ml; about 50 mcg/ml; about 60 mcg/ml; about 65 mcg/ml; or about 70 mcg/ml.

For example, to deliver a 25 microgram (mcg) dose, a composition with a mometasone furoate concentration of 62.5 mcg/milliliter (mL) can be delivered from four sprays of a nasal spray actuator with a spray volume of about 100 microliter (mcL)/spray actuation.

Useful total daily doses of mometasone furoate include, but are not limited to ranges from about 0.04 to about 800 micrograms (“mcg”)/day, about 0.04 to about 400 mcg/day, about 0.04 to about 200 mcg/day, about 0.04 to about 100 mcg/day, about 1 to about 100 mcg/day, about 5 to about 100 mcg/day, about 5 to about 75 mcg/day, about 5 mcg to about 50 mcg/day, from about 10 mcg to about 50 mcg/day, from about 10 mcg to about 45 mcg/day, from about 10 to about 30 mcg/day, from about 40 to about 50 mcg/day, from about 15 mcg to about 25 mcg/day, from about 20 to about 25 mcg/day, about 10 mcg/day, about 15 mcg/day, 20 mcg/day, about 22.5 mcg/day, about 25 mcg/day, about 27.5 mcg/day, about 30 mcg/day about 40 mcg/day, or about 45 mcg/day.

Dosing may be one, two, three or four times daily. Particularly suitable dosing administration is either once daily or twice daily.

Any suitable form of mometasone furoate may be used, including but not limited to mometasone furoate anhydrous and mometasone furoate monohydrate.

Based on the judgment of the attending clinician, the amount of mometasone furoate administered and the treatment regimen used will, of course, be dependent on the age, sex and medical history of the patient being treated, the severity of the specific asthmatic or non-malignant pulmonary disease condition and the tolerance of patient to the treatment regimen as evidenced by local toxicity (e.g., nasal irritation and/or bleeding) and by systemic side-effects (e.g. cortisol level). Cortisol (also referred to as hydrocortisone) is the major natural glucocorticosteroid elaborated by the adrenal cortex.

Suitable diseases that can be treated include corticosteroid-responsive disease of the airway passage ways and lungs which includes those allergic, non-allergic and/or inflammatory diseases of the upper or lower airway passages or of the lungs which are treatable by administering corticosteroids such as mometasone furoate. Typical corticosteroid-responsive diseases include allergic and non-allergic rhinitis, nasal polyps, chronic obstructive pulmonary disease (COPD) as well as non-malignant proliferative and inflammatory diseases of the airways passages and lungs.

The invention is also useful in treating allergic and non-allergic rhinitis as well as non-malignant proliferative and/or inflammatory disease of the airway passages and lungs. Exemplary allergic or inflammatory conditions of the upper and lower airway passages which can be treated or relieved according to various embodiments of the present invention include nasal symptoms associated with allergic rhinitis, such as seasonal allergic rhinitis, intermittent allergic rhinitis, persistent allergic rhinitis and/or perennial allergic rhinitis as well as congestion in moderate to severe seasonal allergic rhinitis patients. Other conditions that may be treated or prevented include corticosteroid responsive diseases, nasal polyps, asthma, chronic obstructive pulmonary disease (COPD), rhinovirus, rhinosinusitis including acute rhinosinusitis and chronic rhinosinusitis, congestion, total nasal symptoms (stuffiness/congestion, rhinorrhea, nasal itching, sneezing) and non-nasal symptoms (itchy/burning eyes, tearing/watery eyes, redness of the eyes, itching of the ears/palate) and nasal blockage associated with sinusitis, fungal induced sinusitis, bacterial based sinusitis.

The term “allergic rhinitis” as used herein means any allergic reaction of the nasal mucosa and includes hay fever (seasonal allergic rhinitis) and perennial rhinitis (non-seasonal allergic rhinitis) which are characterized by seasonal or perennial sneezing, rhinorrhea, nasal congestion, pruritis and eye itching, redness and tearing.

The term “non-allergic rhinitis” as used herein means eosinophilic nonallergic rhinitis which is found in patients with negative skin tests and those who have numerous eosinophils in their nasal secretions.

The term “asthma” as used herein includes any asthmatic condition marked by recurrent attacks of paroxysmal dyspnea (i.e., “reversible obstructive airway passage disease”) with wheezing due to spasmodic contraction of the bronchi (so called “bronchospasm”). Asthmatic conditions which may be treated or even prevented in accordance with this invention include allergic asthma and bronchial allergy characterized by manifestations in sensitized persons provoked by a variety of factors including exercise, especially vigorous exercise (“exercise-induced bronchospasm”), irritant particles (pollen, dust, cotton, cat dander) as well as mild to moderate asthma, chronic asthma, severe chronic asthma, severe and unstable asthma, nocturnal asthma, and psychologic stresses. The invention is particularly useful in preventing the onset of asthma in mammals e.g., humans afflicted with reversible obstructive disease of the lower airway passages and lungs as well as exercise-induced bronchospasm.

The term “non-malignant prolifertive and/or inflammatory disease” as used herein in reference to the pulmonary system means one or more of (1) alveolitis, such as extrinsic allergic alveolitis, and drug toxicity such as caused by, e.g. cytotoxic and/or alkylating agents; (2) vasculitis such as Wegener's granulomatosis, allergic granulomatosis, pulmonary hemangiomatosis and idiopathic pulmonary fibrosis, chronic eosinophilic pneumonia, eosinophilic granuloma and sarcoidoses.

The phrase “therapeutically effective amount” means that amount of a medicament which when administered supplies an amount of one or more pharmaceutically active agents contained therein to provide a therapeutic benefit in the treatment or management of a disease or disease state.

Administration may be accomplished utilizing inhalation devices including but not limited to a nebulizer, a metered pump-spray device, soft mist inhaler and a pressurized metered dosing inhaler. A single pressurized metered dose inhaler may be adapted for oral or nasal inhalation routes simply by switching between an actuator that is designed for nasal delivery and an actuator designed for oral delivery.

Solutions may be administered intranasally by inserting an appropriate device (such as a nasal spray bottle and actuator used to deliver NASONEX® Nasal Spray) into each nostril. Active drug is then expelled from the nasal spray device. Efficacy can be generally assessed in a double blind fashion by a reduction in nasal and non-nasal symptoms (e.g., sneezing, itching, congestion, and discharge). Other objective measurements (e.g., nasal peak flow and resistance) can be used as supportive indices of efficacy. Any suitable pump spray may be used, such as pump sprays used for NASONEX® as sold by Schering-Plough or AFRIN® as sold by Schering-Plough.

Administering mometasone furoate to the surfaces of the airways of asthmatic patients can maximize the therapeutic index. The term “therapeutic index”, as used herein, means the ratio of local efficacy to systemic safety.

Pressurized metered-dose inhalers (“MDI”) contain propellants, for example, chlorofluorocarbon propellants, for example, CFC-11, CFC-12, hydrofluorocarbon propellants, for example, HFC-134A, 1-1FC-227 or combinations thereof, to produce a precise quantity of an aerosol of the medicament contained with the device, which is administered by inhaling the aerosol nasally, treating the nasal mucosa and/or the sinus cavities.

A suitable MDI composition will include a propellant such as 1,1,1,2,3,3,3 heptafluoropropane; an excipient, including but not limited to alcohols, MIGLYOL® 812, MIGLYOL® 840, PEG-400, menthol, lauroglycol, VERTREL®_{—}245, TRANSCUTOL®, LABRAFAC® Hydro WL 1219, perfluorocyclobutane, eucalyptus oil, short chain fatty adds, and combinations thereof; a steroid and optionally a surfactant. MDI's may be prepared by conventional processes such as cold filling or pressure filling.

A “soft-mist” inhaler is a multi-dose, metered aerosol delivery device typically used to deliver aqueous based solution medicaments to the lungs via oral inhalation. The aerosol plume that they create is both slow in velocity and lasts for approximately 6× that of a typical pMDI (e.g. typically 1-2 sec. vs. milliseconds). An example of such a device would be Boehringer Ingelheim's (BI) RESPIMAT® which is currently used to deliver ipatropium bromide to the lungs.

The medicament compositions of the present invention may also be administered utilizing a nebulizer device. Typical commercial nebulizer devices produce dispersions of droplets in gas streams by one of two methods. Jet nebulizers use a compressed air supply to draw liquid up a tube and through an orifice by venturi action and introduce it into a flowing gas stream as droplets suspended therein, after which the fluid is caused to impact one or more stationary baffles to remove excessively large droplets. Ultrasonic nebulizers use an electrically driven transducer to subject a fluid to high-frequency oscillations, producing a cloud of droplets which can be entrained in a moving gas stream; these devices are less preferred for delivering suspensions. For instance, from about 1 to about 4 mL of the mometasone furoate solution may be placed in a plastic nebulizer container and the patient would inhale for 1-30 minutes. The total dosage placed in such a container would be in the range of 0.2 to about 100 mcg.

Also available are hand-held nebulizers which atomize a liquid with a squeeze bulb air supply, but the more widely used equipment incorporates an electrically powered compressor or connects to a cylinder of compressed gas. Although the various devices which are commercially available vary considerably in their delivery efficiency for a given medicament since their respective outputs of respirable droplets are far from identical, any may be used for delivery of the medicaments of the present invention when a prescriber specifies an exact amount of medicament composition which is to be charged to each particular device.

Other embodiments of the present invention provide for pharmaceutical compositions include combinations of a steroid and at least one additional APA, including decongestants, antihistamines, beta agonists and anticholinergics. More particularly, useful combinations of APAs include mometasone furoate and oxymetazoline, mometasone furoate and beta agonists such as formoterol, salmeterol, or indacaterol, mometasone furoate and anticholinergics such as tiotropium, glycopyrrolate, or ipratropium.

One particularly useful combination is mometasone furoate with a decongestant. Examples of suitable decongestants include 1-desoxyephedrine, ephedrine, ephedrine hydrochloride, ephedrine sulfate, naphazoline, naphazoline hydrochloride, oxymetazoline and pharmaceutically acceptable salts thereof, oxymetazoline hydrochloride, phenylephrine, phenylpropanolamine, menazoline, phenylephrine hydrochloride, propylhexedrine, xylometazoline and xylometazoline hydrochloride. Oxymetazoline is a preferred decongestant.

Useful effective total daily amounts of oxymetazoline include from about 5 to about 5000 micrograms (“mcg”)/day, from about 5 to about 2000 mcg/day, about 12.5 to about 1000 mcg/day, about 25 to about 1000 mcg/day, about 12.5 to about 800 mcg/day, about 12.5 to about 600 mcg/day, about 25 to about 500 mcg/day, 25 to about 400 micrograms, about 50 to about 500, about 50 to about 300 mcg/day, from about 50 to about 200 micrograms, from about 100 to about 300 mcg/day, about 100 mcg/day or about 200 mcg/day or about 300 mcg/day in single or divided doses. The total daily dose includes the total amount of drug delivered to both nostrils. Each nostril may receive 1 or 2 sprays.

The mometasone furoate administered to treat disease of the upper or lower airway passages may be used as monotherapy or as adjuvant therapy with for example cromolyn sodium or nedocromil sodium (available from Fisons); bronchodilators such as albuterol (available from Schering Corporation under the PROVENTIL® tradename) or oxymetazoline (available as AFRIN® from Schering-Plough).

Compositions of multiple embodiments of the present invention may include, inter alia, water, auxiliaries and/or one or more of the excipients, such as: suspending agents, e.g., microcrystalline cellulose, sodium carboxymethylcellulose, hydroxpropyl-methyl cellulose; humectants, e.g. glycerin and propylene glycol; acids, bases or buffer substances for adjusting the pH, e.g., citric acid, sodium citrate, phosphoric acid, sodium phosphate as well as mixtures of citrate and phosphate buffers; surfactants, e.g. polysorbate 80; and antimicrobial preservatives, e.g., benzalkonium chloride, phenylethyl alcohol and potassium sorbate.

Depending on the intended application, it may be desirable to incorporate up to about 5 percent by weight, more typically about 0.5 to about 5 weight percent, of an additional rheology-modifying agent, such as a polymer or other material. Useful materials include, without limitation thereto, sodium carboxymethyl cellulose, algin, carageenans, carbomers, galactomannans, hydroxypropyl methylcellulose, hydroxypropyl cellulose, polyethylene glycols, polyvinyl alcohol, polyvinylpyrrolidone, sodium carboxymethyl chitin, sodium carboxymethyl dextran, sodium carboxymethyl starch and xanthan gum. Combinations of any two or more of the foregoing are also useful.

Mixtures of microcrystalline cellulose and an alkali metal carboxyalkylcellulose are commercially available, the mixture presently preferred for use in this invention being sold by FMC Corporation, Philadelphia, Pa. U.S.A. as AVICEL® RC-591. This material contains approximately 89 weight percent microcrystalline cellulose and approximately 11 weight percent sodium carboxymethylcellulose, and is known for use as a suspending agent in preparing various pharmaceutical suspensions and emulsions. The compositions of the present invention may contain at least about 1.0 to about 10 weight percent, or from about 1 to about 4 weight percent of the mixture of the cellulose/carboxyalkylcellulose compound mixture.

A closely related mixture is available from the same source as AVICEL® RC-581, having the same bulk chemical composition as the RC-591, and this material is also useful in the invention. Microcrystalline cellulose and alkali metal carboxyalkylcellulose are commercially available separately, and can be mixed in desired proportions for use in the invention, with the amount of microcrystalline cellulose may be between about 85 and about 95 weight percent of the mixture for both separately mixed and co-processed mixtures.

When the compositions of the invention are intended for application to sensitive mucosal membranes, it may be desirable to adjust the pH to a relatively neutral value, using an acid or base, unless the natural pH already is suitable. In general, pH values about 3 to about 8 are preferred for tissue compatibility; the exact values chosen should also promote chemical and physical stability of the composition. In some instances, buffering agents will be included to assist with maintenance of selected pH values; typical buffers are well known in the art and include, without limitation thereto, phosphate, citrate and borate salt systems.

The compositions may contain any of a number of optional components, such as humectants, preservatives, antioxidants, chelating agents, mucoadhesives, and aromatic substances. Humectants, which are hygroscopic materials such as glycerin, a polyethylene or other glycol, a polysaccharide and the like act to inhibit water loss from the composition and may add moisturizing qualities. Useful aromatic substances include camphor, menthol, eucalyptol and the like, flavors and fragrances. Preservatives are typically incorporated to establish and maintain a freedom from pathogenic organisms; representative components include benzyl alcohol, methylparaben, propylparaben, butylparaben, chlorobutanol, phenethyl alcohol (which also is a fragrance additive), phenyl mercuric acetate and benzalkonium chloride.

Certain aspects of the invention are further described in the following examples. The descriptions of the embodiments of the invention have been presented for purpose of illustration and description. They are not intended to be exhaustive or to limit the invention to the precise forms disclosed, and obviously many modifications and variations are possible in light of the above teaching. The term ‘comprising’ is defined as ‘including but not limited to’.

Percentages are expressed on a weight basis, unless the context clearly indicates otherwise. The mention of any specific drug substance in this specification or in the claims is intended to encompass not only the base drug, but also pharmaceutically acceptable salts, esters, hydrates and other forms of the drug. Where a particular salt or other form of a drug is mentioned, it is contemplated that other salts or forms can be substituted.

### EXAMPLES

### Example 1

### Micellar and Microemulsion Compositions

Various mometasone furoate micellar solutions, such as Compositions A, C, D and G are prepared by measuring each excipient into a beaker the given amounts. If needed, the excipients are melted using a water bath maintained at 65° C. for approximately 30 minutes. The composition is q.s. to 50 g with water and mixed with an overhead/lightening mixer. The MF is added based on the concentration for 50 grams and the solutions are mixed on a lightening mixer for about 7 minutes at 1000 rpm.

Various MF microemulsion solutions, such as Compositions B, E and F, are prepared by measuring each excipient into a beaker the given amounts. If needed, the excipients are melted using a water bath maintained at 65° C. for approximately 30 minutes. The oil component is then added and immediately mixed on an overhead/lightening mixer for 5 minutes at 1000 rpm. The MF is added based on the concentration for 50 grams and the solutions is mixed on a lightening mixer for 20 minutes at 1000 rpm. The composition is q.s. to 50 g with water and mixed with an overhead/lightening mixer.

### Composition A:

1.25 mg anhydrous mometasone furoate g d-alpha tocopheryl polyethylene glycol 1000 succinate
45 g water

### Composition B:

1.25 mg anhydrous mometasone furoate
2.5 g ethyl laurate
10 g polyethylene glycol 660-12 hydroxystearate
37.5 g water

### Composition C:

1.25 mg anhydrous mometasone furoate
5 g polyethylene glycol 12 oxy-stearate
45 g water

### Composition D:

1.25 mg anhydrous mometasone furoate
5 g polyoxyethylene (20) sorbitan monooleate
45 g water

### Composition E:

1.25 mg mometasone furoate monohydrate
2.5 g medium chain triglycerides
10 g Polyethylene glycol 660-12 hydroxystearate
37.5 g water

### Composition F:

6.25 mg anhydrous mometasone furoate
2.5 g medium chain triglycerides
10 g Polyethylene glycol 660-12 hydroxystearate
37.5 g water

### Composition G:

1.25 mg anhydrous mometasone furoate
5 g poloxamer 407
45 g water

### Example 2

### Micellar Solubility Determinations

To determine the solubility of MF in various solutions, various MF solutions are prepared by measuring each excipient into a beaker at a 10% w/w concentration. If needed, the excipients are melted using a water bath maintained at 65° C. for approximately 30 minutes. The composition is q.s. to 50 g with water and mixed with an overhead/lightening mixer. Approximately 50-60 mg of anhydrous mometasone furoate anhydrous is added into each mixture. The solutions are mixed at a low speed on a shaker (Eberbacher) for about 48 to about 72 hours. Approximately 20 mL of each suspension are removed and centrifuged at 1200 rpm for 12 minutes. Approximately 5 mL of the supernatant is filtered using a 0.22 μm syringe filter. A 1 mL sample is taken from each filtered sample, diluted, and assayed using an HPLC. The results are shown in Table 1.

TABLE 1
MF Solubility with 10% Surfactant Solutions in Water

| | | Excipient | |
|---|---|---|---|
| Excipient Trade | Excipient Chemical | Concentration | MF Solubility |
| Name | Name | (w/w) in Water | (μg/mL) |
| Vitamin E TPGS | d-α tocopheryl PEG | 10% | 499 |
| | 1000 succinate | | |
| SOLUTOL ® | Polyethylene glycol | 10% | 184 |
| HS 15 | 660-12 | | |
| | hydroxystearate | | |
| CREMOPHOR ® | Polyoxyl 40 | 10% | 189 |
| RH 40 | hydrogenated castor | | |
| | oil | | |
| TWEEN ® 80 | Polysorbate 80 | 10% | 199 |
| LUTROL ® | Poloxamer 407 | 10% | 27 |
| F127 | | | |

High solubilities are observed from SOLUTOL® HS 15, polysorbate 80, and CREMOPHOR® RH 40. They have approximately 75% by weight PEG esterified onto the lipophilic parts of the molecule.

Vitamin E TPGS has a high solubility of almost 500 μg/g, and CREMOPHOR® RH 40, SOLUTOL HS® 15, and polysorbate 80 had solubilities between approximately 180 and 200 μg/mL. Solutions with 10% polyoxamers and polyvinylpyrrolidones (also highly polar), show ME solubility of less than 30 μg/mL.

### Example 3

### Microemulsion Solubility Determinations

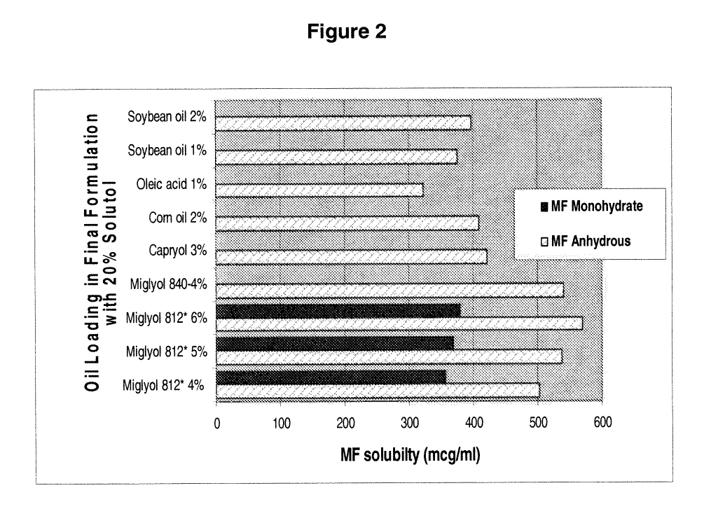

The equilibrium solubility of mometasone furoate is determined in SOLUTOL® HS-15-based microemulsion composition composed of 20% of SOLUTOL® HS-15 with varying amounts of different oils (FIG. 2, Table 2). An excess amount of the drug is added in the solutions after water had been added. The vials is shaken over a 72 hour period. The solutions is centrifuged and filtered at various time intervals prior to analysis of the samples by HPLC. The drug solubility ranged from 300 mcg/ml to 600 mcg/g. Alternative oils are also explored (Table 3).

A concentration of 538 mcg/ml of mometasone furoate was achieved with 5% of MIGLYOL®, 20% w/w of SOLUTOL® HS-15, and 75% phosphate buffer system (PBS). The concentration was ˜519 mcg/ml in a similar composition where PBS was replaced with water. To achieve a concentration of exactly 500 mcg/ml, roughly 5 mg of the drug was weighed and added to a mixture of 2 g of SOLUTOL® HS-15 and 0.5 g of MIGLYOL® 812, that was previously mixed using a magnetic stir bar. After the drug was dissolved in the pre-concentrate, 7.5 g of distilled water was added to the above mix to formulate a microemulsion of mometasone furoate. The drug concentration was determined by HPLC, and was found to be −500 mcg/ml. The oil loaded micellar solution composition of mometasone furoate was physically and chemically stable for at least 2 weeks at elevated temperature and for at least three cycles of freeze thaw.

TABLE 2
Microemulsions with SOLUTOL ® HS-15

| | | | SOLUTOL | Solubilized MF |
|---|---|---|---|---|
| Example | Oil | Oil w/w | HS 15 w/w ® | (mcg/ml) |
| 1 | MIGLYOL ® | 4% | 20% | 502 |
| | 812 | | | |
| 2 | MIGLYOL ® | 5% | 20% | 538 |
| | 812 | | | |
| 4 | MIGLYOL ® | 4% | 20% | 540 |
| | 840 | | | |
| 5 | CAPRYOL ® | 3% | 20% | 422 |
| 6 | Corn oil | 3% | 20% | 408 |
| 7 | Oleic acid | 2% | 20% | 323 |
| 8 | Soybean oil | 1% | 20% | 375 |
| 9 | Soybean oil | 2% | 20% | 397 |

TABLE 3
Microemulsions with Ethyl Laurate

| | Oil | | Surfactant | MF Solubility |
|---|---|---|---|---|
| Oil excipient | w/w | Surfactant | w/w | (μg/mL) |
| Ethyl laurate | 5% | Polyethylene glycol | 20% | 540 |
| | | 660-12 | | |
| | | hydroxystearate | | |
| Ethyl laurate | 5% | POLOXAMER ® 407 | 10% | 90 |

### Example 4

### Cyclodextrin Solutions

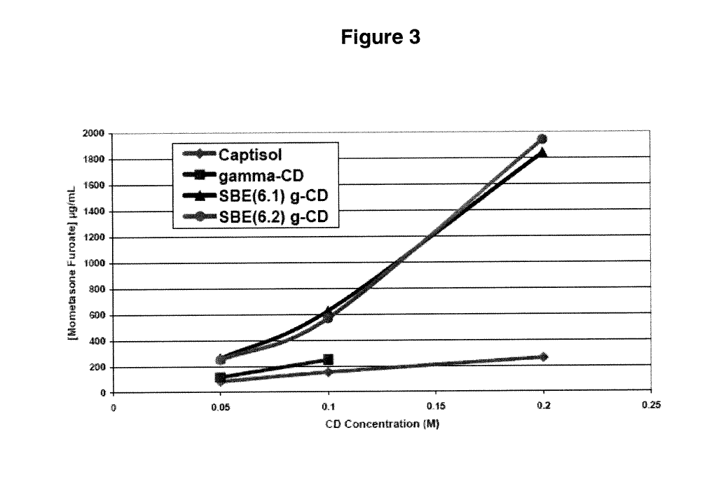

Compositions of mometasone furoate 6 mg/ml were prepared in duplicate with a 0.05, 0.01, and 0.2 M of four different cyclodextrins: an unsubstituted gamma cyclodextrin, a sulfobutyl ether (SBE) beta cyclodextrin (Captisol), and two substituted SBE gamma cyclodextrins. Only 0.05 and 0.1M cyclodextrins were evaluated for the unsubstituted gamma cyclodextrin. All compositions were prepared in a 3 mM citrate buffer at a pH of 4.5. The compositions were placed on a roller mixer (Stuart Scientific SRT233 rpm rise/fall 16 mm) protected from light and mixed for about three days. After the three day equilibration, the compositions were filtered using a 0.22 micrometer PVDF syringe filter and analyzed by HPLC. The data is shown in FIG. 3 and Table 4, indicating that the two more substituted gamma sulfobutyl ether cyclodextrins (6.1, 6.2) solubilized greater amounts of MF.

TABLE 4
Concentration of MF in solutions with cyclodextrins

| | Cyclodextrin (CD) Conc. | | |
|---|---|---|---|
| | 0.05M | 0.1M | 0.2M |
| Captisol (SBE beta-CD) | 84 | 152 | 261 |
| unsubstituted gamma-CD | 117 | 249 | |
| SBE(6.1) gamma-CD | 262 | 626 | 1838 |
| SBE(6.2) gamma-CD | 250 | 569 | 1940 |

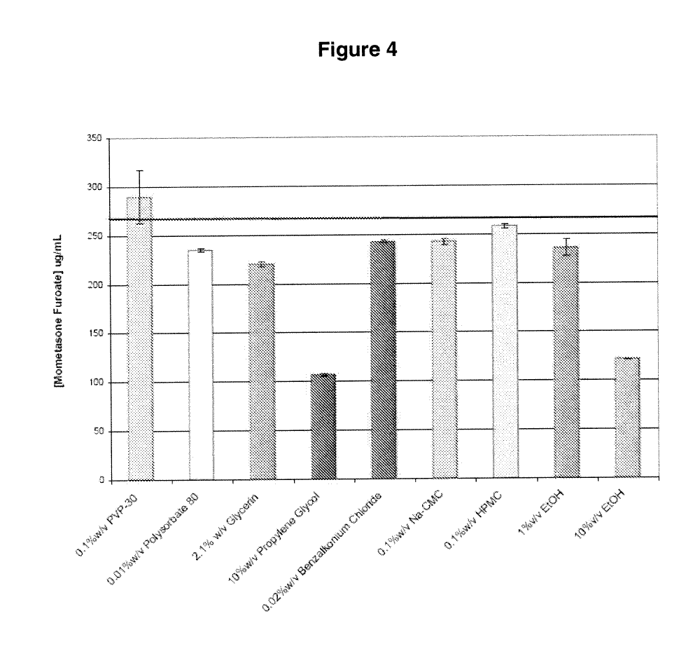

A stock solution of 0.2 M Captisol (SBE beta cyclodextrin) was prepared. Table 5 and FIG. 4 list the effect of various additives on the MF solubility of this solution:

TABLE 5

| Additive | MF Solubility. mcg/mL |
|---|---|
| 0.1% w/v PVP-30 | 290 |
| 0.01% w/v Polysorbate 80 | 235 |
| 2.1% w/v Glycerin | 220 |
| 10% w/v Propylene Glycol | 107 |
| 0.02% w/v Benzalkonium | 243 |
| Chloride | |
| 0.1% w/v Na-CMC | 242 |
| 0.1% w/v HPMC | 258 |
| 1% v/v EtOH | 236 |
| 10% v/v EtOH | 121 |

### Example 5

### MDI Compositions

Metered dose Inhaler (MDI) compositions are prepared by the following method: oleic acid is added and dissolved in ethanol in an appropriate pressurized vessel. The MF is then added to the alcohol mixture and dissolved with high speed agitation or using a homogenizer. The drug solution is then filled into MDI cans and the propellants are added by pressure filling.

| Excipient | MDI 1 (mg/g) | MDI 2 (mg/g) |
|---|---|---|
| MF | 0.84 | 0.42 |
| Ethanol (200 proof) | 140.00 | 120.00 |
| Oleic acid | 0.005 | 0.01 |
| HFA 134a | Fill to 1.0 g | — |
| HFA 227 | — | Fill to 1.0 g |

### Example 6

### Clinical Study

A recently concluded clinical study conducted by Schering-Plough compared the systemic exposure of MF from (A) a commercial NASONEX® suspension nasal spray and (B) a MF solution delivered by the same device as used for NASONEX®. The design and methods of the clinical trial.

### Clinical Study Design

The clinical study conducted was a part-randomized, crossover, open-label study of MF conducted in healthy adult volunteers. A total of 12 male or female subjects were enrolled. On each of the study periods each subject received 1 of the 2 treatments as shown below in each period according to their randomized treatment sequences.

Treatment A MF 200 μg administered as an aqueous suspension from a NASONEX® Nasal Spray (2 alternating sprays×50 μg per burst in each nostril)

Treatment B MF 200 μg administered as a solution using the pump spray from NASONEX® (2 sprays×50 μg per burst in each nostril)

Each of the 4 sprays from the NASONEX® Nasal Spray was delivered to alternating nostrils to minimize drip/run-off and maximize nasal cavity deposition (e.g. left nostril, right nostril, left nostril, right nostril).

Blood Specimen Collection

At hours 0 (predose), 0.5, 1, 1.5, 2, 4, 6, 8, 10 and 12 on Day 1 of each of the 3 study periods, 6.5-mL blood samples (6-mL sample plus 0.5 mL discard) were collected from an indwelling venous catheter from each subject for pharmacokinetic analysis of MF.

Assay Method

MF concentrations in human plasma samples were determined by using a high-pressure liquid chromatographic-tandem mass spectrometric method. This proprietary method, that was developed and validated by PPD, Richmond, Va., was specific for MF, i.e. metabolites of MF were not detected. The assay sample volume was 1.00 mL of human plasma. The lower limit of quantification was 0.25 pg/mL.

Results

FIG. 1 compares the systemic exposure of MF following administration of the suspension and solution compositions at the same dose from the same device. The MF exposure is determined by measuring the area under the curve (AUC) of the plasma levels at the time points that were measured. When comparing a NASONEX® suspension (upper line-circles) to a mometasone furoate solution (lower line-squares), the MF blood levels from an MF solution were significantly greater (about 1000%) than from a NASONEX® suspension even though both compositions were delivered at the same dose and with the same type of device. At 12 hours after dosing, concentrations of MF after administration as a solution were still approximately 600% higher than the MF concentrations observed after administration of MF in the form of a suspension.

1-96. (canceled)

97. A pharmaceutical composition comprising a mometasone furoate aqueous solution suitable for inhalation, wherein the concentration of mometasone furoate is from about 0.02 mcg/ml to about 500 mcg/ml.

98. The composition of claim 97, wherein the concentration of the mometasone furoate is from about 25 mcg/ml to about 75 mcg/ml.

99. The composition of claim 97, wherein the concentration of the mometasone furoate is from about 5 mcg/ml to about 50 mcg/ml.

100. The composition of claim 97, wherein the composition comprises at least one co-solvent.

101. The composition of claim 100, wherein the composition further comprises at least one co-solvent selected from the group consisting of propylene glycol, polyethylene glycol 300, polyethylene glycol 400, ethanol, N—N dimethylacetamide, N-methyl-2-pyrolidone and glycerol and combinations thereof.

102. The composition of claim 100, wherein the at least one co-solvent is present in an amount from about 5 to about 15% by weight.

103. The composition of claim 97, wherein the composition comprises at least one surfactant.

104. The composition of claim 97, wherein the composition comprises at least one surfactant and at least one oil.

105. The composition of claim 104, wherein the at least one oil is present in an amount from about 0.01 to about 40% by weight.

106. The composition of claim 104, wherein the at least one oil is present in an amount from about 1 to about 10% by weight.

107. The composition of claim 97, wherein the composition comprises at least one oil selected from the group consisting of short, medium or long chain monoglycerides, diglycerides or triglycerides and combinations thereof.

108. A pharmaceutical drug product comprising an inhalation device and the composition of claim 97.

109. The pharmaceutical composition of claim 97 further comprising a surfactant with a hydrophilic-lipophilic balance value from about 4 to about 18; optionally an oil; and water.

110. The composition of claim 97, wherein the composition comprises a microemulsion.

111. The composition of claim 97, wherein the composition comprises a micellar composition.

112. A method of treating an airway disease comprising administering the composition of claim 1 once daily to the upper or lower airway passages; wherein the total daily dose of mometasone furoate is from about 0.04 to about 200 micrograms.

113. The method of claim 112; wherein the total daily dose of mometasone furoate is from about 5 to about 50 micrograms of mometasone furoate.

114. The method of claim 112, wherein the airway disease is asthma, chronic obstructive pulmonary disease, sinusitis, allergic rhinitis or nasal polyposis.

115. A pharmaceutical composition comprising an aqueous solution suitable for inhalation comprising mometasone furoate in a concentration from about 5 mcg/ml to about 100 mcg/ml; at least one surfactant in a concentration from about 0.01 to about 20% by weight; optionally at least one oil in a concentration from about 0.01 to about 20% by weight; and water.

116. A pharmaceutical composition comprising a solution suitable for inhalation, comprising mometasone furoate; at least one excipient having a hydrophilic moiety and a hydrophobic moiety; and water.
